(19) United States
(12) Patent Application Publication
(10) Pub. No.: US 2026/0256983 A1
(43) Pub. Date: Sep. 3, 2026

(54) REGENERATIVE BONE PUTTY SYSTEMS, METHODS, AND DEVICES

(71) Applicant: ThoraGenix Innovations, Inc., Tampa, FL (US)

(72) Inventors: Frederick J. Thabet, Boca Raton, FL (US); Ravi Kanagala, Boca Raton, FL (US); Keri George, Calgary (CA)

(73) Assignee: ThoraGenix Innovations, Inc., Tampa, FL (US)

(21) Appl. No.: 19/344,465

(22) Filed: Sep. 29, 2025

Related U.S. Application Data
(60) Provisional application No. 63/701,179, filed on Sep. 30, 2024.

(51) Int. Cl. A61L 27/24; A61L 27/44; A61L 27/54

(52) U.S. Cl. CPC A61L 27/24; A61L 27/446; A61L 27/54; A61L 2430/02

(57) ABSTRACT

Systems, methods, and devices disclosed herein include a bone healing system comprising a regenerative bone healing structure formed at a target area of a damaged bone. The regenerative bone healing structure includes a regenerative putty and one or more reinforcement structure(s). The reinforcement structure(s) are configured to be positioned around the target area of the damaged bone. The regenerative putty has a composition including a matrix material and a bone growth compound. Also, the regenerative putty is operable to fill a space formed by the reinforcement structure. The method(s) include positioning the reinforcement structure(s) around the target area such that the reinforcement structures define an open lattice space operable to receive the putty. The method(s) also include positioning the regenerative putty at the open lattice space such that the regenerative putty fills the target area. Additionally, the method(s) include causing bone growth at the target area with the regenerative putty.

### CROSS-REFERENCE TO RELATED APPLICATION

This application claims priority to U.S. Provisional Application Ser. No. 63/701,179, filed Sep. 30, 2024, and titled “REGENERATIVE BONE PUTTY SYSTEMS, METHODS, AND DEVICES,” the entirety of which is incorporated by reference herein.

### BACKGROUND

Traditional bone repair treatments have always been associated with safety issues and other complications. Various bone fractures, defects, and voids can occur from injury, surgery, or other sources of damage to the human body. Additionally, nonunion is a common complication after attempting to heal a bone fracture, and can cause improper or incomplete bone healing. This is a major challenge for orthopedic surgeons worldwide because of the high incidence rate and difficulties in achieving successful bone repair. Bone voids and bone defects are the main complications of nonunion. The significant amount of time required for the bone healing process to occur prolongs the window during which complications can arise and increases the likelihood of nonunion. Sometimes bones do not fully heal and can remain vulnerable to infection or a second fracture. Furthermore, some bone fractures are difficult to access to perform any bone healing/alignment procedures.

It is with these observations in mind, among others, that various aspects of the present disclosure were conceived and developed.

### SUMMARY

Systems, methods, and devices disclosed herein address the aforementioned problems. For instance, a bone repair system can include a reinforcement structure configured to be positioned around a target area of a damaged, degenerated, demineralized, genetically deformed, idiopathically deformed, infected, iatrogenically destabilized, or traumatized bone. The bone repair system can also include a regenerative putty having a composition including a matrix material and a bone growth compound. The regenerative putty can be operable to fill a space formed by the reinforcement structure.

In some examples, the matrix material can include a bovine collagen matrix. The composition of the regenerative putty can also include at least one of a bioglass catalyst, bone minerals, or bone salts. Additionally, the reinforcement structure can include one or more of a fiber mesh, a strut, or a cage. Furthermore, the composition can cause the regenerative putty to be inductive to blood cells. The composition can have hemostatic properties which can make the regenerative putty irrigation-resistant with respect to blood. Moreover, the reinforcement structure can be transparent to x-rays. The bone repair system can also include a putty applicator tool (e.g., a ThoraSpreader) having an elongated body and a functional end used for positioning the regenerative bone putty at the target area.

In some scenarios, a bone healing system includes a regenerative putty having a composition including a matrix material and a bone growth compound. The bone healing system can also include one or more reinforcement structures forming a regenerative bone healing structure configured to be positioned around a target area of a damaged bone, the regenerative bone healing structure forming a space operable to receive the regenerative putty.

In some instances, the one or more reinforcement structures can involve a plurality of different reinforcement structures including at least two of a fiber mesh, a strut, or a cage. Additionally, the one or more reinforcement structures can augment any sternal, rib, or thoracic skeletal fixation hardware. The regenerative bone healing structure can be formed by a first layer being the fiber mesh and a second layer being the strut or the cage. Also, the regenerative putty can be positioned between the fiber mesh and a bone surface at the target area. Additionally or alternatively, the regenerative putty can be positioned between the fiber mesh and the strut or the cage. Moreover, the regenerative bone healing structure can form an open lattice space with a plurality of reinforcement structures which receives the regenerative putty.

In some examples, the regenerative bone healing structure can secure the regenerative putty into a cavity formed into a bone of the target area. Also, a shape of the regenerative bone healing structure can correspond to a shape of a complex fracture at the target area. Moreover, bone healing at the target area can be optimized by the composition of the regenerative putty pulling blood cells into the target area while the regenerative putty is secured in place by the one or more reinforcement structures.

In some scenarios, a method to heal a damaged portion of bone can include positioning one or more reinforcement structures around a target area of a damaged bone, the one or more reinforcement structures defining an open lattice space operable to receive a regenerative putty. The method can also include positioning the regenerative putty at the open lattice space such that the regenerative putty fills the target area, the regenerative putty having a composition including a matrix material and a bone growth compound which, with the one or more reinforcement structures, forms a regenerative bone healing structure. Additionally, the method can include causing bone growth at the target area with the regenerative putty. Furthermore, causing the bone growth can include using an inductive characteristic of the composition of the regenerative putty to pull blood cells into the regenerative bone healing structure. Moreover, positioning the one or more reinforcement structures around the target area can include applying a fiber mesh around the target area and then applying a strut or a cage around the fiber mesh. Additionally, the regenerative putty can be used to augment a thoracic skeletal fixation system hardware.

### BRIEF DESCRIPTION OF THE DRAWINGS

FIG. 1 illustrates an example bone repair system including a regenerative putty and one or more reinforcement structure(s).

FIG. 2 illustrates example bone repair systems including different target area configurations.

FIG. 3 illustrates an example bone repair system to repair various section(s) of the thoracic skeletal system.

FIG. 4 illustrates an example method to use a bone repair system to heal a damaged bone, which can be performed by the system(s) depicted in FIGS. 1-3.

### DETAILED DESCRIPTION

It will be appreciated that numerous specific details are set forth in order to provide a thorough understanding of the examples described herein. However, it will be understood by those of ordinary skill in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

The systems, methods, and devices disclosed herein include a bone repair system comprising a regenerative putty and/or a reinforcement structure for positioning the regenerative putty at a target location. In some instances, the regenerative putty can be used without mesh and can be applied directly on the compromised bone as a bone void filler. Further, the regenerative putty can be used to augment thoracic skeletal fixation hardware and systems. This disclosed technology can be used to heal broken or fractured bones, diseased bones, bone voids, bone defects, and/or bone deterioration. Furthermore, the bone repair system can form an irrigation resistant structure with a natural antibiotic effect.

Additional benefits and advantages of the disclosed technology will become apparent from the detailed description below.

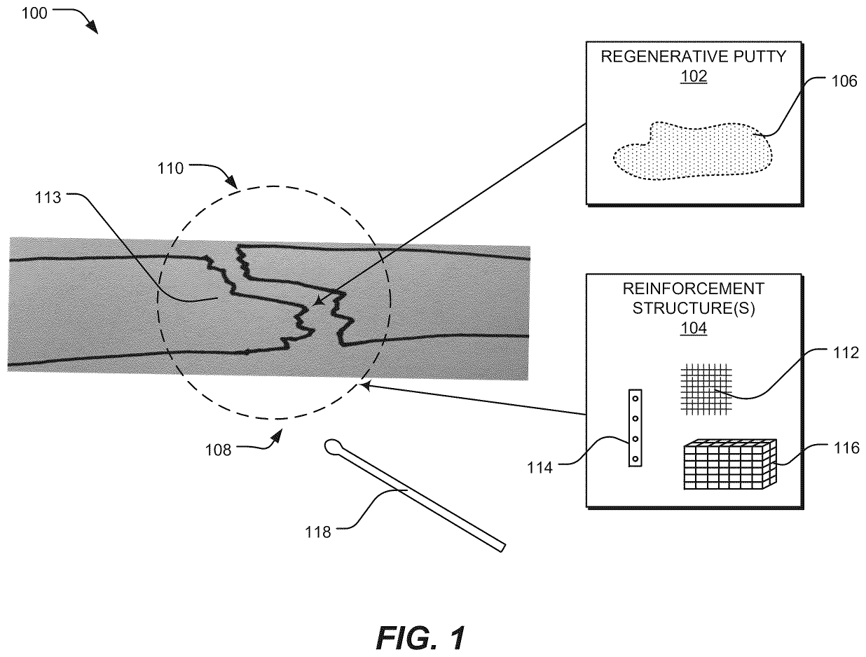

FIG. 1 illustrates an example bone repair system 100 including a regenerative putty 102 and/or a reinforcement structure 104. The regenerative putty 102 can include a particular composition 106 to optimize a bone healing process 108 performed with the bone repair system 100. For instance, the regenerative putty 102 can be formed of a matrix material, such as demineralized bone matrix (DBM) and/or a collagen matrix (e.g., a bovine collagen matrix), and/or a bioglass. These components can make the regenerative putty 102 conductive and/or inductive such that cells (e.g., blood cells) are drawn into the regenerative putty 102. Moreover, the composition 106 of the regenerative putty 102 may be irrigation-resistant with respect to blood. The regenerative putty 102 can include various bone growth compounds (e.g., osteoblasts, stem cells, exosomes, and so forth).

In some cases, the regenerative putty 102 can have various properties and attributes. For instance, the regenerative putty 102 can be considered an IRM (Irrigation Resistant Matrix) in that the regenerative putty 102 can be a collagen base matrix. This means it can be molded before application and pressed into the bone defect, void, or area of incised bone to help control bleeding from the bone during and after the procedure. Due to the collagen base, the regenerative putty 102 may avoid being wash out or being dislodged from the bone during or after the surgery due to excessive bleeding. Therefore, the IRM and/or collagen base of the regenerative putty 102 can prevent the bone from bleeding during the procedure. In addition, irrigating the wound during surgery may not cause the matrix to be dislodged either. Additionally, the regenerative putty 102 can have superior handling characteristics because the regenerative putty 102 putty can be in a collagen base matrix. For instance, by being made from either bovine, porcine or plant based collagen, the regenerative putty 102 can be highly moldable, flowable and can be “pressed” into areas of bone that have defects or voids. Moreover, the regenerative putty 102 can have superior healing qualities. For example, the regenerative putty 102 can be impregnated with all the necessary minerals that help bone to grow such as Beta Tricalcium Phosphate (TCP), Hydroxyapatite, Vitamin D, Phosphorus, Magnesium, Copper, Calcium, Potassium, Zinc, Boron, Vitamin K, Various peptides, Silicon, Fluoride, Calcium Phosphate Salts, and/or any combination thereof. These minerals can combine to create an optimal environment that allows optimal bone environment for cells to be attracted to the putty as an osteoconductive scaffold. Furthermore, the regenerative putty 102 can include growth factors. For instance, various growth factors can be added to the putty that act as catalysts to encourage cellular activity to increase as the bone putty moves through the osteogenesis process. These growth factors can include Peptides, Bone Morphogenic Proteins, BioGlass™ (e.g., a surface reactive glass and/or ceramic material that is biocompatible and/or degradable in body fluid), other growth factors, and/or any combination thereof. These growth factors can help create the optimal environment that allows bone growth cells to be attracted to the putty as an osteoinductive scaffold. Also, the regenerative putty 102 can have material porosity, such as a particular pore size that allows for optimal bone growth to occur. This can be a pore size range between 400 to 500 microns, or another pore size. The regenerative putty 102 can be specifically engineered to maximize this pore size range. Additionally, the regenerative putty 102 can provide bone reinforcement. For example, defects often occur in bone, especially in patients that have multiple comorbidities prior to surgery. These defects can contribute to poor bone quality and compromised stability of the bone. When the regenerative putty 102 is applied to the defects, the regenerative putty 102 can help reinforce this poor bone and initiate the healing process. Moreover, the regenerative putty 102 can have injectability. For instance, because of the flowable nature of the regenerative putty 102, it can be injected into various bony defects that are more recessed and immediately non-accessible to the surgeon's direct application abilities with his/her fingers.

Also, the regenerative putty 102 can specifically be used for sternal bone healing, to augment sternal fixation hardware, to support sternal bone haemostasias, and/or to reinforce sternal bone integrity. In the ribs, the regenerative putty 102 can be used for rib fracture repair, rib fracture healing, rib fracture stabilization, rib fracture haemostasias, and to augment rib fracture hardware. Moreover, various methods of implementing and applying the regenerative putty 102 to the bone surfaces can result in improved healing, as well.

In some scenarios, the reinforcement structure 104 can include one or more (e.g., a plurality) of various structures used for positioning and/or securing the regenerative putty 102 at a target area 110. The reinforcement structure 104 can include a fiber mesh 112 which can be applied to a portion of bone 113. Then, a strut 114 and/or a cage 116 can be placed over the fiber mesh 112. With the fiber mesh 112, the strut 114 and/or the cage 116 in place, open space within the fiber mesh 112 can be filled with the regenerative putty 102. The strut 114 can include a substantially elongated and/or flat body. Furthermore, a layer of the regenerative putty 102 can be applied to the target area 110 prior to application of the fiber mesh 112, the strut 114, and/or the cage 116. For instance, a method of using the bone repair system 100 can include applying a first layer of the regenerative putty 102 before applying the reinforcement structure(s) 104 and applying a second layer of the regenerative putty 102 after applying the reinforcement structure(s) 104. In some scenarios, the bone healing process 108 can include positioning the fiber mesh 112 under and/or adjacent to the strut 114, and/or filling space within and around the fiber mesh 112 and the strut 114 with the regenerative putty 102. Additionally or alternatively, the reinforcement structure(s) 104 can include the cage 116, which can be positioned around the target area 110 and can have open spaces which can become filled with the regenerative putty 102. The bone healing process 108 can include using any one of or any combination of the fiber mesh 112, the strut 114, and/or the cage 116. The cage 116 can be positioned adjacent and/or at least partly surrounding the fiber mesh 112 and/or the strut 114. The reinforcement structure(s) 104 can also include an interbody cage which can be a cylindrical, crescent-shaped, hollow, and/or porous metallic device (e.g., a BAK cage).

The reinforcement structure(s) 104 can, together or separately, form an open lattice structure at least partly surrounding the target area 110 and/or integrating into the target area 110. A size, shape, and/or various dimensions of the open lattice structure can correspond to a size, shape, and/or dimension(s) of the target area 110 (e.g., an exposed bone side, a fracture length, or so forth). The open lattice structure formed by the reinforcement structure(s) 104 can have an irregular shape formed to correspond to a complex bone defect being healed by the bone repair system 100.

In some examples, the bone repair system 100 can be non-load bearing or only partially load bearing. For instance, the regenerative putty 102 can operate to fill space (e.g., within the reinforcement structure(s) 104 and/or the target area 110) such that bone growth cells from the surrounding area are pulled into the filled space. Additionally or alternatively, the reinforcement structure 104 can provide some structural/load bearing support for the skeletal system (e.g., by preventing bone movement/sliding).

In some instances, the reinforcement structure 104 can include a rigid material such as titanium, polyether ether ketone (PEEK), and/or biologics. In some scenarios, the reinforcement structure(s) 104 can be formed of a material that is transparent to x-rays and/or other imaging technology. Additionally or alternatively, the reinforcement structure 104 can be formed of a flexible material, such as the fiber mesh 112.

In some examples, the bone repair system 100 can include a putty applicator tool 118. The putty applicator tool can be a handheld device with an elongated gripping body and a functional end, such as a trowel, rod, scoop, or other shape for applying and/or manipulating the regenerative putty 110 into position.

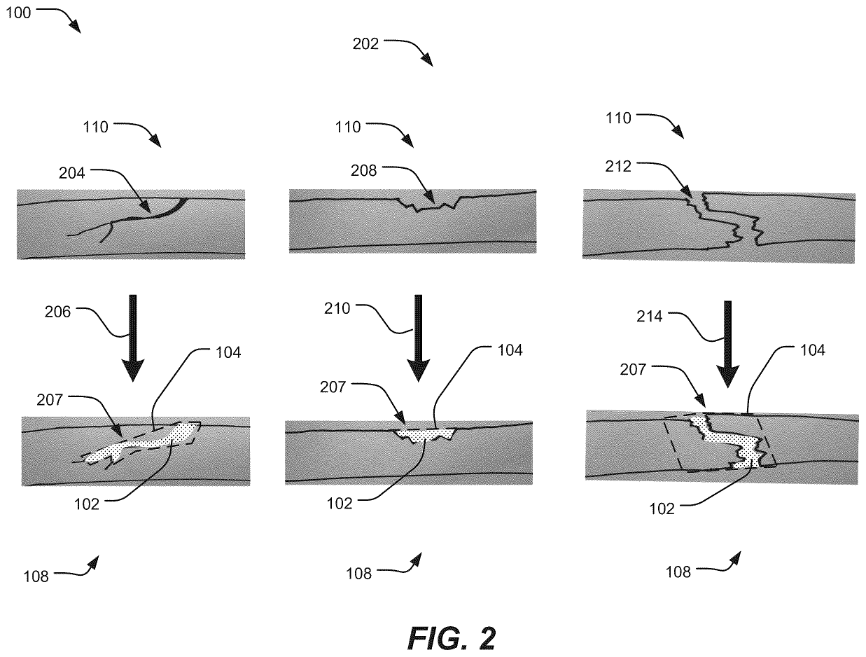

FIG. 2 depicts an example bone repair system 100 showing one or more different target area configuration(s) 202, which can form at least a portion of the bone repair system 100 depicted in FIG. 1.

In some scenarios, the target area 110 can include a first target area 204 which can involve a hairline or linear fracture. A first bone repair procedure 206 can be performed at the first target area 204 to create a regenerative bone healing structure 207 around the first target area 204. For instance, the first bone repair procedure 206 can include placing the regenerative putty 102 in, around, and/or over the hairline fracture. The first bone repair procedure 206 can also include securing (e.g., with adhesives, wires, screws, or so forth, etc.) a strut 114 across at least a portion of the hairline fracture and/or positioning a fiber mesh 112 at least partly across the hairline fracture (e.g., beneath and/or over the strut 114). Additionally or alternatively, the regenerative putty 102 can be placed over the reinforcement structures 104 and/or can be pushed through openings in the reinforcement structures 104 (e.g., openings in the fiber mesh 112). As such, the regenerative bone healing structure 207 can include a combination of the regenerative putty 102 and the reinforcement structure(s) 104.

In some examples, the target area 110 can include a second target area 208 which can involve a greenstick fracture in which a crack is formed into the outer surface of the bone and/or a portion of bone is missing from the outer surface. A second bone repair procedure 210 can be performed at the second target area 208 to create the regenerative bone healing structure 207 around the second target area 208. For instance, the second bone repair procedure 210 can include placing the regenerative putty 102 in, around, and/or over the greenstick fracture. The second bone repair procedure 210 can also include securing (e.g., with adhesives, wires, screws, or so forth, etc.) the fiber mesh 112 and/or the cage 116 across at least a portion of the greenstick fracture to keep the regenerative putty 102 in place (e.g., filling a void of the greenstick fracture). Additionally or alternatively, the regenerative putty 102 can be placed over the reinforcement structures 104 and/or can be pushed through openings in the fiber mesh 112 and/or the cage 116. As such, the regenerative bone healing structure 207 can include a combination of the regenerative putty 102 and the reinforcement structure(s) 104.

In some examples, the target area can include a third target area 212 which can involve a transverse, oblique, and/or comminuted fracture. A third bone repair procedure 214 can be performed at the third target area 212 to create the regenerative bone healing structure 207 around the third target area 212. For instance, the third bone repair procedure 214 can include placing the regenerative putty 102 in, around, and/or over the transverse fracture. The third bone repair procedure 214 can also include securing (e.g., with adhesives, wires, screws, or so forth, etc.) the fiber mesh 112, the strut(s) 114, and/or the cage 116 across at least a portion of the transverse fracture to keep the regenerative putty 102 in place. Additionally or alternatively, the regenerative putty 102 can be placed over the reinforcement structures 104 and/or can be pushed through openings in the fiber mesh 112 and/or the cage 116. As such, the regenerative bone healing structure 207 can include a combination of the regenerative putty 102 and the reinforcement structure(s) 104.

Furthermore, the target area(s) 110 disclosed herein can include a simple fracture, a compound fracture, a greenstick fracture, a hairline fracture, a buckle fracture, an avulsion fracture, a growth plate fracture, a compression fracture, a comminuted fracture, a segmental fracture, a transverse fracture, a spiral fracture, an oblique fracture, and/or any combination thereof. Moreover, a bone repair procedure can include any of the operations of the first bone repair procedure 206, the second bone repair procedure 210, the third bone repair procedure 214, and/or any combination thereof.

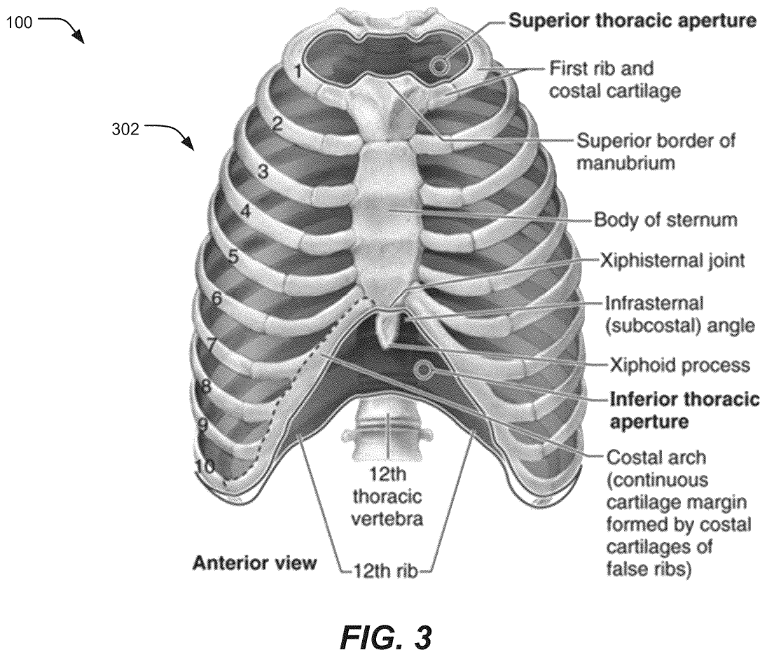

FIG. 3 depicts an example bone repair system 100 to repair a target area 110 at a thoracic skeletal system 302, which can form at least a portion of the bone repair system 100 depicted in FIG. 1.

The bone repair system 100 can perform the bone healing process 108 to heal one or more sections of the thoracic skeletal system 302. For instance, the target area 110 can include one or more of a superior border of manubrium, a body of the sternum, a xiphisternal joint, an infrasternal (subcostal) angle, a xiphoid process, an edge of an inferior thoracic aperture, an edge of a superior thoracic aperture, any of ribs 1-12, a thoracic vertebra, combinations thereof, and/or any other portion of the thoracic cage.

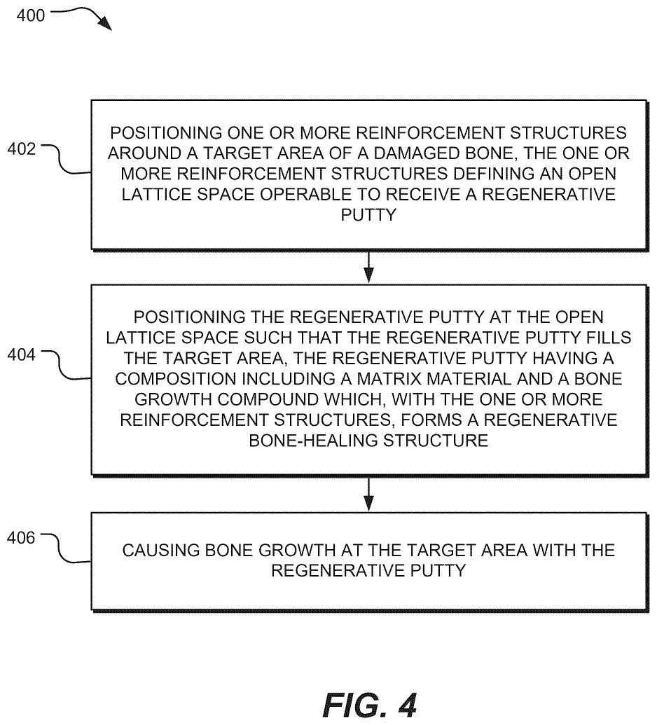

FIG. 4 depicts an example method 400 to use the bone repair system 100 to heal a damaged bone, which can be performed by any of the system(s) 100 depicted in FIGS. 1-3.

In some examples, a first operation 402 of the method 400 can position one or more reinforcement structures around a target area of a damaged bone. The one or more reinforcement structures can define an open lattice space operable to receive a regenerative putty. At operation 404, the method 400 can position the regenerative putty at the open lattice space such that the regenerative putty fills the target area. The regenerative putty can have a composition including a matrix material and a bone growth compound which, with the one or more reinforcement structures, forms a regenerative bone healing structure. At operation 406, the method 400 can cause bone growth at the target area with the regenerative putty.

It is to be understood that the specific order or hierarchy of steps in the method(s) depicted throughout this disclosure are instances of example approaches and can be rearranged while remaining within the disclosed subject matter. For instance, any of the operations depicted throughout this disclosure may be omitted, repeated, performed in parallel, performed in a different order, and/or combined with any other of the operations depicted throughout this disclosure.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A bone healing system comprising:
a reinforcement structure configured to be positioned around a target area of a damaged, degenerated, demineralized, genetically deformed, idiopathically deformed, infected, iatrogenically destabilized, or traumatized bone; and
a regenerative putty having a composition including a matrix material and a bone growth compound, the regenerative putty being operable to fill a space formed by the reinforcement structure.

2. The system of claim 1,
wherein,
the matrix material includes a bovine collagen matrix.

3. The system of claim 1,
wherein,
the composition of the regenerative putty includes at least one of a bioglass catalyst, bone minerals, or bone salts.

4. The system of claim 1,
wherein,
the reinforcement structure includes one or more of a fiber mesh, a strut, or a cage.

5. The system of claim 1,
wherein,
the composition causes the regenerative putty to be inductive to blood cells.

6. The system of claim 1,
wherein,
the composition has hemostatic properties which make the regenerative putty irrigation-resistant with respect to blood.

7. The system of claim 1,
wherein,
the reinforcement structure is transparent to x-rays.

8. The system of claim 6, further comprising:
a putty applicator tool having an elongated body and a functional end.

9. A bone healing system comprising:
a regenerative putty having a composition including a matrix material and a bone growth compound; and
one or more reinforcement structures forming a regenerative bone healing structure configured to be positioned around a target area of a damaged bone, the regenerative bone healing structure forming a space operable to receive the regenerative putty.

10. The system of claim 9,
wherein,
the one or more reinforcement structures involves a plurality of different reinforcement structures including at least two of a fiber mesh, a strut, or a cage, or
the one or more reinforcement structures augment sternal, rib, or thoracic skeletal fixation hardware.

11. The system of claim 10,
wherein,
the regenerative bone healing structure is formed by a first layer being the fiber mesh and a second layer being the strut or the cage.

12. The system of claim 11,
wherein,
the regenerative putty is positioned between the fiber mesh and a bone surface at the target area.

13. The system of claim 11,
wherein,
the regenerative putty is positioned between the fiber mesh and the strut or the cage.

14. The system of claim 9,
wherein,
the regenerative bone healing structure forms an open lattice space with a plurality of reinforcement structures which receives the regenerative putty.

15. The system of claim 9,
wherein,
the regenerative bone healing structure secures the regenerative putty into a cavity formed into a bone of the target area.

16. The system of claim 9,
wherein,
a shape of the regenerative bone healing structure corresponds to a shape of a complex fracture at the target area.

17. The system of claim 9,
wherein,
bone healing at the target area is optimized by the composition of the regenerative putty pulling blood cells into the target area while the regenerative putty is secured in place by the one or more reinforcement structures.

18. A method to heal a damaged portion of bone, the method comprising:
positioning one or more reinforcement structures around a target area of a damaged bone, the one or more reinforcement structures defining an open lattice space operable to receive a regenerative putty; and
positioning the regenerative putty at the open lattice space such that the regenerative putty fills the target area, the regenerative putty having a composition including a matrix material and a bone growth compound which, with the one or more reinforcement structures, forms a regenerative bone healing structure; and
causing bone growth at the target area with the regenerative putty.

19. The method of claim 18,
wherein,
causing the bone growth includes using an inductive characteristic of the composition of the regenerative putty to pull blood cells into the regenerative bone healing structure.

20. The method of claim 19,
wherein,
positioning the one or more reinforcement structures around the target area includes applying a fiber mesh around the target area and then applying a strut or a cage around the fiber mesh.

21. The method of claim 18,
wherein,
the regenerative putty is used to augment a thoracic skeletal fixation system hardware.
